(19) United States
(12) Patent Application Publication
(10) Pub. No.: US 2004/0081625 A1
(43) Pub. Date: Apr. 29, 2004

(54) Nasal formulations for the treatment of allergies

(75) Inventors: Stefan A. Sharpe, Jersey City, NJ (US); Joel A. Sequeira, Edison, NJ (US); Varda Sandweiss, Forest Hills, NJ (US); Mark C. Giordano, Roseland, NJ (US); Saeed Chaudhry, Clifton, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 10/690,233

(22) Filed: Oct. 21, 2003

Related U.S. Application Data
(60) Provisional application No. 60/420,013, filed on Oct. 21, 2002.

(51) Int. Cl. A61L 9/04; G01N 21/00; G01N 21/75

(52) U.S. Cl. 424/45; 436/164

(57) ABSTRACT

Disclosed is a method of determining the formation of a secondary structure of a thixotropic formulation, said method comprising a) placing an amount of said thixotropic formulation on a transparent object; b) capturing an image of said thixotropic formulation by back-scattered light by using a particle vision and measurement probe; c) converting said image to a video image; d) analyzing said video image to determine the amount of time it takes for the formation of said secondary structure within said thixotropic formulation, and novel formulations concerning the same.

### CROSS REFERENCE TO RELATED APPLICATION

This application claims benefit of priority to U.S. Patent Application Serial No. 60/420,013 filed Oct. 21, 2002 which is incorporated by reference.

This invention relates to methods of determining the thixotropic properties of formulations useful for treating of corticosteroid-responsive diseases of the upper airway passages, such as allergic rhinitis, by intra-nasally administering to said passages an amount of mometasone furoate effective for treating such diseases.

Mometasone furoate is a corticosteroid approved for topical dermatologic use to treat inflammatory and/or pruritic manifestations of corticosteroid-responsive dermatoses. The compound may be prepared in accordance with the procedures disclosed in U.S. Pat. Nos. 4,472,393, 4,731,447, 4,873,335, 5,837,699 and 6,127,353, which U.S. Patents are hereby incorporated by reference in their entirety. Mometasone is a topically active steroid which is not readily bioavailable would provide a therapeutic advantage over other topically active corticosteroids that are more systematically bioavailable and it would also be superior to any corticosteroid orally administered by the oral swallowing of, for example, a solution, tablet or capsule. It is commercially available as a spray for intra-nasal administration under the name of Nasonex AQ®.

The aqueous suspensions of mometasone furoate monohydrate may contain from about 0.01 to 10.0 mg, preferably 0.1 to 10.0 mg of mometasone furoate monohydrate per gram of suspension. The aqueous suspension compositions according to the present invention may contain, inter alia, water, auxiliaries and/or one or more of the excipients, such as: suspending agents, e.g., microcrystalline cellulose, sodium carboxymethylcellulose, hydroxpropyl-methyl cellulose; humectants, e.g. glycerin and propylene glycol; acids, bases or buffer substances for adjusting the pH, e.g., citric acid, sodium citrate, phosphoric acid, sodium phospate as well as mixtures of citrate and phosphate buffers; surfactants, e.g. Polysorbate 80; and antimicrobial preservatives, e.g., benzalkonium chloride, phenylethyl alcohol and potassium sorbate.

Nasonex AQ contains the following ingredients in roughly the following amounts. These amounts may be varied and are set forth for exemplary purposes.

TABLE 1

| INGREDIENT | MG/G |
|---|---|
| Mometasone Furoate Monohydrate | *0.1-10 |
| Micronized Cellulose and | 15-25 |
| Carbothymethylcellulose Sodium NF | |
| 65 cps | |
| Glycerin USP | 18-24 |
| Citric Acid Monohydrate USP | 18-24 |
| Sodium Citrate Dihydrate USP | 2.6-3.0 |
| Polysorbate 80 NF | 0.05-0.15 |
| Benzalkonium Chloride Solution NF | 0.15-0.25 |
| Phenylethyl Alcohol USP | 2-3 |
| Water Purified USP q.s. ad | 1000 |

The microcrystalline cellulose and carboxymethylcellulose sodium are present as suspending agents. The glycerin serves as a humectant. The glycerin also imparts a moisturizing effect on the mucous membrane and renders the formulation physiologically isoosmotic. The sodium citrate dihydrate and citric acid monohydrate serve as a buffering system to achieve and maintain a target pH of about 4.5. The polysorbate 80 serves as a wetting agent and aids in the dispersion of the mometasone furoate monohydrate in water. The benzalkonium chloride and phenyethyl alcohol serve as preservative agents. Alternatively, the formulation may be phenylethyl alcohol free to produce a formulation without fragrance. Finally, the purified water serves as the vehicle.

A common problem with spray administration is that the structure of body cavities and parts does not typically facilitate retention of the applied formulation. This is particularly the case for aqueous-based nasal spray formulations, which must have sufficient fluidity to be dispensed by a pump device or a squeeze-type spray bottle, but which can simply drain from the nose or pass through the nose and into the pharynx while, or immediately after, being sprayed. Moreover, due to ciliation of the nasal passages and movement of air through the nose, even materials applied in particulate form, such as by a pressurized metered dose inhaler or a powdered drug inhaler, are rapidly cleared from the nose. Several of the possible active agents or other formulation components have a quite unpleasant taste, so it is desirable to minimize the amount of the formulation which is not retained within the nose for at least the minimum time required to obtain the desired effect. Due to swallowing of much of the formulation which enters the oropharyngeal area, a large portion of the active agent introduced into the nose is generally rendered unavailable for its intended use. As such, it would be beneficial to have nasal sprays that do not suffer from such infirmities, such as Nasonex AQ®.

Typically, these suspensions are thixotropic fromulations. Thixotropy can be defined as an isothermal, reversible change in magnitude of the rheological properties of a system. Thixotropic formulations can be described as systems that respond to stress or strain with a series of viscoelastic phenomena. These responses are either in the linear region where the micro-structure responds linearly in response to the applied stress or strain but does not change, or in the non-linear region where the microstructure does change in response to the imposed stresses and strains, but reversibly. This reversible micro-structural change takes time to transpire due to local spatial rearrangement of the formulation components.

Typically, conventional rotational viscometry methods measured only the extent or magnitude thixotropy in Pascals/second. The rate of formation, i.e, the need for secondary structure formation, however, has not been previously determined. In addition, the use of laser back scattered light has not been employed. Accordingly, there exists a need for methods of determining the rheological characteristics of thixotropic suspensions for administration to the passages of the upper airways having acceptable properties that have not previously been determined.

### SUMMARY OF THE INVENTION

The present invention discloses a method of determining the formation of a secondary structure of a thixotropic formulation, said method comprising a) placing an amount of said thixotropic formulation on a transparent object; b) capturing an image of said thixotropic formulation by back-scattered light by using a particle vision and measurement probe; c) converting said image to a video image; d) analyzing said video image to determine the amount of time it takes for the formation of said secondary structure within said thixotropic formulation, and novel formulations concerning the same.

### DETAILED DESCRIPTION OF THE INVENTION

Disclosed is a method of determining a secondary structure of a thixotropic formulation, said method comprising a) placing an amount of said thixotropic formulation on a transparent object; b) capturing an image of said thixotropic formulation by back-scattered light by using a particle vision and measurement probe; c) converting said image to a video image; d) analyzing said video image to determine the amount of time it takes for the formation of said secondary structure within said thixotropic formulation. This invention is directed to a novel method for the determination of the formation of secondary structure in thixotropic formulations.

As set forth above, thixotropy can be defined as an isothermal, reversible change in magnitude of the rheological properties of a system. The magnitude of thixotropy of commercially available nasal sprays has been analyzed. The rheological study was performed using an AR1000 Rheometer by TA Instruments. The geometry used to perform the analysis was a 40 mm steel cone with dimensions of 2 degrees, 0 minutes, and 51 seconds. The truncation of the geometry was 50 microns. The sample was shaken for 5 to 10 seconds before being actuated into a beaker. The sample was poured from the beaker to the Peltier plate for analysis. The sample was allowed to equilibrate to 25 degrees Celsius before performing the rheological analysis. An Up/Down experiment with a conditioning step was used to perform the analysis of the sample at 25 degrees Celsius. The conditioning step occurred before the actual Up/Down experiment began; the conditioning step was performed for 2 minutes. The Up Step was set for a shear rate ramp of 0/sec to 100/sec over an 8 minute period. Fifty data points were taken for the Up Step. The Down Step was set for a shear rate ramp of 100/sec to 0/sec in an 8 minute period. Fifty data points were also taken for the Down Step. The results of this analysis are set forth below in Table 2.

TABLE 2
THIXOTROPY MAGNITUDE

| Product | Thixotropy (Pa/s)^{a} |
|---|---|
| Nasacort ® | 1.07 |
| Rhinocort ® | 1.14 |
| Flonase ® | 6.73 |
| Vancenase ® | 1.17 |
| Nasonex AQ ® | 22.9 |

^{a}Average of three (3) units

As is evident from the above results, Nasonex AQ, available from Schering Corporation, displayed a significantly greater magnitude of thixotropy as compared to other nasal sprays. For instance, Nasonex AQ is roughly 3.1 times as thixotropic as Flonase®.

One aspect of the present invention is the ability to capture at isothermal conditions the initial change in magnitude of the rheological properties of a system, e.g. the speed of flow. Then the second time-dependent element of thixotropy is captured which is the formation of a secondary structure. This formation of secondary structure is captured through back-scattered light by Particle Vision and Measurement (“PVM”) 700 Probe available from Lasentec Laser Sensor Technology, Inc. and consequent video image analysis, also from Lasentec PVM Laser Sensor Technology, Inc.

This method was capable in distinguishing between thixotropic materials depending on the magnitude of their thixotropy and the rate of secondary structure formation. The more thixotropic materials e.g Nasonex AQ formed secondary structure faster and consequently stopped flowing sooner than the less thixotropic materials.

The methods of determining the point of secondary structure in the formulations of the present invention have several advantages. First, the effect of the concentration of the structure building elements in the formulation, e.g., for example, microcrystalline cellulose and carboxymethylcellulose sodium NF, on the time of formation of secondary structure may be ascertained. The analytical methods of the present invention have the ability to capture the formation of the secondary structure and correlate the time for its occurrence in relation to the flow properties of the formulation such as yield stress, viscosity and thixotropy. Further, the methods of the present invention also have the ability to study the effect of homogenization on the time of formation of secondary structure in the finished product of the present invention. These methods are also useful in determining the acceptability of thixotropic pharmaceutical formulations, particularly for nasal administration.

### EXAMPLE 1

To carry out one aspect of the present invention, orient the PVM probe parallel to a bench top in a manner sufficient to allow the sample flow to appear on the videoscreen of the instrument in a downward fashion. Take a clean glass slide and orient it in the vertical position so that it is perpendicular to the PVM probe. Place the glass slide directly on to the PVM probe. Spray the sample directly on to the side of the glass slide opposite to the PVM probe. Allow the sample to flow freely down the glass side while the PVM records the image sequence. The software will record the flow time in seconds. The formation of the secondary structure is observed as a decrease in the flow of the sample with the formation of regions of higher density and thicker consistency, that eventually will form a thick and immobile matrix. The total time of flow of the material down the slide is the time elapsed before the formation of the secondary structure.

TABLE 3
THIXOTROPY RATE

| | MeanTime for Secondary |
|---|---|
| Product | Structure Formation (seconds) |
| Nasacort ® | N/A^{a} |
| Rhinocort ® | N/A^{a} |
| Flonase ® | N/A^{a} |
| Vancenase ® | N/A^{a} |
| Nasonex AQ ® | 64 |

^{a}Not applicable. Samples did not exhibit secondary structure formation throughout the experiment and eventually dripped off the slide

Surprisingly, Nasonex AQ forms a secondary structure of a time period of about 28 to about 85 seconds, which is a significant improvement relative to other nasal products. For instance, Flonase®, Nasacort®, Rhinocort® and Vancenase® did not form a secondary structure within a reasonable period of time.

The foregoing descriptions of various embodiments of the invention are representative of various aspects of the invention, and are not intended to be exhaustive or limiting to the precise forms disclosed. Many modifications and variations undoubtedly will occur to those having skill in the art. It is intended that the scope of the invention shall be fully defined solely by the appended claims.

What is claimed is:

1. A method of determining the formation of a secondary structure of a thixotropic formulation, said method comprising:
a) placing an amount of said thixotropic formulation on a transparent object;
b) capturing an image of said thixotropic formulation by back-scattered light by using a particle vision and measurement probe;
c) converting said image to a video image;
d) analyzing said video image to determine the amount of time it takes for the formation of said secondary structure within said thixotropic formulation.

2. The method of claim 1, wherein the thixotropic formulation is a pharmaceutically acceptable formulation for intra-nasal admimistration.

3. The method of claim 2, wherein said thixotropic formulation comprises mometasone furoate.

4. The method of claim 3, wherein said thixotropic formulation comprises at least about 10 mg/g of at least one suspending agent.

5. The method of claim 4, wherein said suspending agent comprises microcrystalline cellulose and carboxymethylcellulose sodium NF.

6. The method of claim 3, wherein said thixotropic formulation has a pH of about 3.5 to about 7.

7. The method of claim 6, wherein said thixotropic formulation has a pH of about 4.5.

8. The method of claim 3, wherein said thixotropic formulation further comprises a humectant.

9. The method of claim 3, wherein said thixotropic formulation further comprises a humectant.

10. The method of claim 3, wherein said thixotropic formulation does not contain alcohol.

11. The method of claim 3, wherein said secondary structure forms in about 25 to about 85 seconds.

12. The method of claim 3, wherein said secondary structure forms in at least about 25 seconds.

13. The method of claim 2, wherein said thixotropic formulation comprises beclomethasone diproprionate.

14. The method of claim 12, wherein said thixotropic formulation contains less than about 2% of micronized cellulose and carbothymethylcellulose sodium.

15. The method of claim 2, wherein said thixotropic formulation triamcinolone acetonide.

16. The method of claim 14, wherein said thixotropic formulation contains less than about 2% of micronized cellulose and carbothymethylcellulose sodium.

17. The method of claim 2, wherein said thixotropic formulation comprises budesonide.

18. The method of claim 16, wherein said thixotropic formulation contains less than about 2% of micronized cellulose and carbothymethylcellulose sodium.

19. The method of claim 2, wherein said thixotropic formulation comprises fluticasone proprionate.

20. The method of claim 16, wherein said thixotropic formulation contains less than about 2% of micronized cellulose and carbothymethylcellulose sodium NF.
